(19) United States
(12) Patent Application Publication
(10) Pub. No.: US 2008/0260823 A1
(43) Pub. Date: Oct. 23, 2008

(54) ORALLY DISINTEGRATING TABLET COMPRISING GLYCOPYRROLATE FOR TREATING SIALORRHEA

(75) Inventor: Larry Dillaha, Atlanta, GA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Sciele Pharma, Inc., Atlanta, GA (US)

(21) Appl. No.: 11/738,452

(22) Filed: Apr. 20, 2007

(51) Int. Cl. A61K 9/20; A61K 31/4015; A61P 1/00

(52) U.S. Cl. 424/464; 514/424

(57) ABSTRACT

The invention provides an orally disintegrating tablet comprising a therapeutically effective amount of glycopyrrolate for treating sialorrhea, as well as a kit comprising the orally disintegrating tablet, prescribing information, and a container. The invention also provides a method of treating sialorrhea in a patient comprising administering the orally disintegrating tablet comprising a therapeutically effective amount of glycopyrrolate to the patient.

### BACKGROUND OF THE INVENTION

Glycopyrrolate, the active pharmaceutical ingredient in Robinul® tablets, Robinul® Forte tablets and Robinul® injection, is a quaternary ammonium compound having the chemical name 3-[(cyclopentylhydroxyphenylacetyl)oxy]-1,1-dimethylpyrrolidinium bromide. Glycopyrrolate is an anticholinergic and antimuscarinic agent. Glycopyrrolate is indicated for use as a preoperative antimuscarinic to reduce salivary, tracheobronchial, and pharyngeal secretions. See Physicians' Desk Reference (57th ed., Medical Economics Co., 2003). Glycopyrrolate also is used to treat the symptoms of some neurological disorders. In particular, glycopyrrolate may be used to reduce excessive saliva that may pool in the mouth or leak out. This condition is known as sialorrhea (persistent or excessive drooling).

Persistent or excessive drooling beyond the age of three years is considered abnormal. Such drooling may be found in individuals with neurological dysfunction or motor deficits (e.g., cerebral palsy, peripheral neuromuscular disease, facial paralysis, and mental retardation) and other conditions such as esophageal cancer. Drooling causes impairment of speech, feeding and swallowing problems, upper respiratory congestion, and choking upon aspiration. Control of drooling is important in preventing choking and gagging in persons with posterior drooling.

Sialorrhea may cause a range of physical and psychosocial complications, including perioral chapping, dehydration, odor, and social stigmatization, that can be devastating for patients and their families. Current recommendations for treating sialorrhea include a clinical team of primary health care providers, speech pathologists, occupational therapists, dentists, orthodontists, neurologists, and otolaryngologists. Treatment options range from conservative (i.e., observation, postural changes, and biofeedback) to more aggressive measures, such as radiation, surgical intervention, and medication.

The ingestion of anticholinergic medications, such as glycopyrrolate, is effective in reducing drooling, but their systemic use may be limited by side effects. In addition, it may be difficult to administer these medications to patients who have trouble swallowing.

Accordingly, there is a need in the art for an easily taken and easily administrable solid preparation comprising glycopyrrolate for patients (e.g., aged or pediatric patients) who have difficulty in swallowing.

There is further a need in the art for a solid preparation of glycopyrrolate that more specifically addresses the problem of sialorrhea so as to reduce the side effects associated with systemic administration of an anticholinergic and antimuscarinic compound.

### BRIEF SUMMARY OF THE INVENTION

In one aspect, the invention is an orally disintegrating tablet comprising a therapeutically effective amount of glycopyrrolate (i.e., an amount of glycopyrrolate effective for treating sialorrhea). The therapeutically effective amount of glycopyrrolate may be from about 1 milligram (mg) to about 10 mg. In one embodiment, the glycopyrrolate may be from about 1 mg to about 2 mg. Further, the orally disintegrating tablet disintegrates or dissolves upon contact with saliva in about five minutes or less. In another embodiment, the orally disintegrating tablet disintegrates or dissolves upon contact with saliva in about three minutes or less, and in some embodiments the orally disintegrating tablet disintegrates or dissolves upon contact with saliva in about one minute or less.

In another aspect, the invention is a method of treating sialorrhea in a patient. The method includes identifying a patient demonstrating sialorrhea and administering an orally disintegrating tablet comprising a therapeutically effective amount of glycopyrrolate to the patient. The orally disintegrating tablet generally has the same characteristics as stated above regarding the amount of glycopyrrolate and disintegration time. In one embodiment, the therapeutically effective amount of glycopyrrolate is administered from one to three times daily. In another embodiment, the patient is older than three years of age. In yet another embodiment, the method of the present invention is useful for treating patients suffering from cancer about the face, neck, or esophagus, or for treating patients suffering from a neurological dysfunction or disorder. Neurological dysfunctions or disorders may include, for example, Parkinson's Disease, stroke, cerebral palsy, amyotrophic lateral sclerosis, and mental retardation.

In yet another aspect, the invention is a method of treating sialorrhea in a patient by providing a kit including one or more orally disintegrating tablets having a therapeutically effective amount of glycopyrrolate, prescribing information, and a container. The prescribing information instructs a patient or the caregiver of a patient regarding administering the therapeutically effective amount of glycopyrrolate. The orally disintegrating tablet generally has the same characteristics as stated above regarding the amount of glycopyrrolate and disintegration time. Furthermore, the prescribing information included with the kit instructs a patient or the caregiver of the patient regarding the appropriate patient age and frequency of administration. In one embodiment, the kit included in the method of the present invention is useful for treating sialorrhea in patients suffering from facial paralysis or cancer about the face, neck, or esophagus. In another embodiment, the kit included in the method of the present invention is useful for treating sialorrhea in patients suffering from a neurological function or disorder, including Parkinson's Disease, stroke, cerebral palsy, amyotrophic lateral sclerosis, and mental retardation.

### DETAILED DESCRIPTION OF THE INVENTION

The invention provides an orally disintegrating tablet that comprises, as an active ingredient, glycopyrrolate, and methods of treating sialorrhea (excessive drooling) by administrating the orally disintegrating tablet to a patient.

Glycopyrrolate exists in four distinct stereoisometric forms due to the presence of two chiral centers in the glycopyrrolate molecule. One of the two enantiomeric pairs of diastereomers of glycopyrrolate is (R,R)-glycopyrrolate and (S,S)-glycopyrrolate, and the other enantiomeric pair is (R,S)-glycopyrrolate and (S,R)-glycopyrrolate. The glycopyrrolate suitable for use in the present invention may be a mixture of two or more of the four stereoisomers. Alternatively, glycopyrrolate may be used in the form of one isolated enantiomer.

Enantiomerically enriched glycopyrrolate may also be used. Enantiomerically enriched (S,S)-glycopyrrolate, (R,R)-glycopyrrolate, (S,R)-glycopyrrolate, and (R,S)-glycopyrrolate, and methods of their preparation, are described in U.S. Pat. No. 6,063,808, U.S. Pat. No. 6,204,285, International Patent Application Publication WO 98/00109, and International Patent Application Publication WO 98/00132.

The orally disintegrating tablet of the invention dissolves or disintegrates rapidly with saliva, thus eliminating the need for chewing the tablet, swallowing an intact tablet, or taking the tablet with liquids (e.g., water). The orally disintegrating tablet preferably has an disintegration time of five minutes or less (e.g., four minutes or less, three minutes or less, two minutes or less, or 1.5 minutes or less), more preferably one minute or less (e.g., 55 seconds or less, 50 seconds or less, 45 seconds or less, 40 seconds or less, or 35 seconds or less), and desirably 30 seconds or less (e.g., 25 seconds or less, 20 seconds or less, 15 seconds or less, 10 seconds or less, or 5 seconds or less).

Since the orally disintegrating tablet disintegrates or dissolves in the mouth, preferably the taste of the glycopyrrolate and other accessory ingredients is masked. Taste masking can be achieved by any suitable manner, including the addition of flavoring agents and/or sweeteners, wet granulation or roller compaction with other excipients to minimize the presented surface area of the glycopyrrolate, spray drying, sealing with a suitable coating material (e.g., hydroxypropyl methylcellulose, ethylcelluclose, methacrylates, Kollicoat™, and polyvinylpyrrolidone), and encapsulation.

For example, the active ingredient (e.g., glycopyrrolate) can be microencapsulated in one or more acrylic polymers (e.g., Eudragit E, Eudragit L-55, Eudragit RL) or gelatin. Additionally, fine granules of the drug (e.g., glycopyrrolate) and disintegrant (e.g., low substituted hydroxypropyl cellulose) can be coated with a water insoluble polymer (e.g., ethylcellulose) to mask the taste of the drug.

Suitable flavoring agents include, for example, strawberry flavor, grape flavor, cherry flavor, cotton candy flavor, mint flavor, or other suitable flavor. The flavoring agent or mixtures thereof typically is present in an amount of about 0.0001 wt. % to about 5 wt. %.

Suitable sweeteners include, for example, sugars such as sucrose, lactose, and glucose, cyclamate and salts thereof, saccharin and salts thereof, ammonium glycyrrhizinate, and aspartame. The sweetener or mixtures thereof typically is present in an amount of about 0.001 wt. % to about 70 wt. %.

The orally disintegrating tablets can be prepared by any methods well known in the art of pharmacy, for example, using methods such as those described in Remington: The Science and Practice of Pharmacy, 21st Edition. Philadelphia, Pa.: Lippincott Williams & Wilkins, 2005. Such methods include the step of bringing into association the active ingredient with a carrier (i.e., a pharmaceutically acceptable carrier) which constitutes one or more accessory ingredients. Such accessory ingredients include those conventional in the art, such as, fillers (e.g., polyhydric alcohols, such as mannitol, sorbitol, and xylitol, or mixtures thereof); binders (e.g., acacia, tragacanth, gelatin, sucrose, pre-gelatinized starch, starch, sodium alginate, methylcellulose, sodium carboxymethyl cellulose, ethyl cellulose, hydroxypropylmethyl cellulose, polyvinylpyrrolidone, and polyacrylamide); diluents; disintegrants; lubricants (e.g., talc, magnesium stearate, mineral oil, and mixtures thereof); colorants; flavoring agents; preservatives (e.g., alkyl hydroxbenzoates or salts thereof, such as methyl, ethyl, propyl, and/or butyl hydroxybenzoates; sorbic acid or a salt thereof; benzoic acid or a salt thereof, and mixtures thereof); and wetting agents.

In particular, the orally disintegrating tablet can be prepared by processes, including, but not limited to, freeze-drying or lyophilization, tablet molding, direct compression, spray drying, sublimation, mass extrusion, and microencapsulation. Such processes are well known in the art (see, e.g., U.S. Pat. Nos. 5,178,878, 5,501,861, 5,587,172, 5,607,697, 5,616,344, and 5,622,719, 5,683,720, 5,720,974, 5,807,577, 5,837,285, 5,939,091, 6,036,974, 6,316,026, and 6,696,085; and Bhaskaran et al., Indian Pharmacist, 1(2): 9-12 (2002)).

Examples of orally disintegrating tablets include Zydis™ tablets (R. P. Scherer, Inc.) (see, e.g., Seager, J. Pharm. Pharmacol., 50: 375-382 (1998)), Orasolv™ and Durasolv™ (CIMA Labs, Inc.) (see, e.g., U.S. Pat. Nos. 5,178,878, 6,024,981, 6,221,392, and 6,365,182), Flashtab™ (Ethypharm), WOWTAB™ (Yamanouchi Pharma Technologies), Ziplets™ (Eurand), and Fast Melt™ (Elan Corp.).

Freeze-drying or lyophilization results in tablets that are highly porous with a high specific surface area. The tablets dissolve rapidly and demonstrate improved absorption and bioavailability. Methods of preparing orally disintegrating tablets by lyophilization are known in the art, such as those disclosed in U.S. Pat. Nos. 5,955,488, 6,063,802, and 6,010,719; Corveleyn et al., and Int. J. Pharm., 152: 215-225 (2000); and Jaccard et al., Ann. Pharm. Fr., 43(2): 123-131 (1985).

Molded tablets disintegrate rapidly and can offer a pleasant taste due to the inclusion of water soluble sugars. Methods of preparing orally disintegrating tablets by tablet molding are well known in the art. For example, one method involves moistening a powder blend comprising the drug with a hydroalcoholic solvent, pressing the resulting solution into a mold plate to form a wetted mass (compression molding), and removing the solvent by air drying. In another method, molded forms are prepared using a heat-molding process (see, e.g., U.S. Pat. No. 5,466,464). An additional method of tablet molding is no-vacuum lyophilization, which involves the evaporation of a solvent from a drug solution or suspension at standard pressure (see, e.g., U.S. Pat. No. 5,298,261).

Spray drying results in highly porous, fine powders, which can be formed into orally disintegrating tablets. Methods of preparing orally disintegrating tablets by spray drying are known in the art (see, e.g., U.S. Pat. Nos. 5,587,180, 5,595,761, 5,635,210, and 5,807,576). For example, a formulation containing the drug, a support agent for the matrix (e.g., hydrolyzed gelatin and unhydrolyzed gelatin), a bulking agent (e.g., mannitol), and a disintegrant (e.g., sodium starch glycolate or crosscarmellose) can be spray dried to yield a porous powder. Disintegration and dissolution can be further enhanced by adding an acid (e.g., citric acid) or an alkali (e.g., sodium bicarbonate) before spray drying.

Sublimation results in the presence of a porous structure in the tablet matrix. Methods of preparing orally disintegrating tablets by sublimation are known in the art (see, e.g., U.S. Pat. Nos. 3,885,023, 4,134,943, 5,720,974, and 5,762,961, and Koizumi et al., Int. J. Pharm., 152: 127-131 (1997)). As an example, volatile ingredients (e.g., ammonium bicarbonate, ammonium carbonate, benzoic acid, camphor, hexamethonium tetramine, naphthalene, phtalic anhydride, urea, and urethane) can be used in the tableting process, wherein the volatile material is removed by sublimation and a porous matrix is left behind.

The technique of direct compression can be applied to orally disintegrating tablets if disintegrants and/or sugar-based excipients are included in the tableting process. Methods of preparing orally disintegrating tablets by direct compression are known in the art. Microcrystalline cellulose, cross-linked carboxymethyl cellulose sodium, cross-linked polyvinyl pyrrolidone, and low or partially substituted hydroxypropyl cellulose absorb water and swell due to capillary action, making them effective disintegrants in the preparation of orally disintegrating tablets (see, e.g., Bi et al., Chem. Pharm. Bull., 44(11): 2121-2127 (1996); and Watanbe et al., Biol. Pharm. Bull, 18(9): 1308-1310 (1995)). Agar powder also can be used as a disintegrant because the powder absorbs water and swells considerably without forming a gel at physiological temperatures (see, e.g., Ito et al., Chem. Pharm. Bull, 44(11): 2132-2136 (1996)). Sugar-based excipients, such as dextrose, fructose, isomalt, maltitok, maltose, mannitol, sorbitol, starch hydrolyse, polydextrose, and xylitol, also can be used in the direct compression process to impart aqueous solubility and sweetness. Furthermore, the fast disintegration of tablets can be achieved by incorporating effervescent disintegrating agents, which generate gas.

Suitable effervescent disintegrating agents include agents that evolve gas by means of a chemical reaction that takes place upon exposure of the effervescent disintegrating agent to water and/or to saliva in the mouth. The reaction is most often a result of the reaction of a soluble acid source and an alkali monocarbonate or carbonate source, which produces carbon dioxide gas upon contact with water or saliva. The acid sources can be any that are safe for human consumption including citric acid, tartic acid, amalic acid, fumeric acid, adipic acid, and succinic acid. Carbonate sources include dry solid carbonate and bicarbonate salt, such as sodium bicarbonate, sodium carbonate, potassium bicarbonate, potassium carbonate, and magnesium carbonate. Reactants that generate oxygen or other gases that are safe for human consumption also are suitable.

Administration of the orally disintegrating tablet of the invention is useful in the treatment of neurological dysfunctions, diseases, and disorders that include sialorrhea (excessive drooling) as a symptom. Examples include, but are not limited to, neurological disorders, Parkinson's Disease, stroke, motor deficits (e.g., cerebral palsy, peripheral neuromuscular disease, amyotrophic lateral sclerosis, facial paralysis, and mental retardation), and other conditions, such as cancer about the face, neck, or esophagus. Accordingly, the invention provides a method of treating sialorrhea in a patient comprising administering an orally disintegrating tablet comprising a therapeutically effective amount of glycopyrrolate to the patient.

The orally disintegrating tablet comprising a therapeutically effective amount of glycopyrrolate can be administered alone or in combination with other active agents (e.g., other anti-drooling drugs).

A therapeutically effective amount refers to the amount of a drug or pharmaceutical agent that elicits a biological or medical response of a tissue, system, or animal (e.g., a mammal, such as a human) that is being sought by a researcher, veterinarian, medical doctor, or clinician. With regard to treating sialorrhea, a therapeutically effective amount is the amount of glycopyrrolate that will prevent or alleviate excessive drooling in a patient (e.g., a human patient).

In general, a suitable dose of a therapeutically effective amount of glycopyrrolate or a pharmaceutically acceptable salt thereof for administration to a patient will be between approximately 0.0005 to 300 mg per kilogram body weight of the recipient per day, preferably between approximately 0.0005 and 50 mg/kg/day, and most preferably between approximately 0.001 to 10 mg/kg/day. A typical dose of a therapeutically effective amount of glycopyrrolate or pharmaceutically acceptable salt thereof in human patients is about 1-100 mg/day, preferably about 1-50 mg/day, and more preferably about 1-10 mg/day.

Glycopyrrolate can be administered as two, three, four, five, six or more sub-doses administered separately at appropriate intervals throughout the day in unit dosage forms (e.g., the orally disintegrating tablet of the invention). The typical amount of glycopyrrolate in the orally disintegrating tablet of the invention is about 1-10 mg (e.g., 1 mg, 2 mg, 3 mg, 4 mg, 5 mg, 6 mg, 7 mg, 8 mg, 9 mg, or 10 mg), preferably about 1-5 mg, and most preferably about 1-2 mg. Each orally disintegrating tablet contains a therapeutically effective amount of glycopyrrolate.

In one embodiment, dosing of a therapeutically effective amount of glycopyrrolate is one milligram three times daily, for example, in the morning, early afternoon, and at bedtime. After the initial dosage of glycopyrrolate, an adequate dosage for maintenance of one milligram twice per day is often suitable. Alternatively, glycopyrrolate may be administered two or three times daily with two milligrams per dose. Typically, with all glycopyrrolate dosing schedules, the time periods between administrations are equal.

In another aspect of the invention, the invention is a kit comprising one or more orally disintegrating tablets, prescribing information, and a container. The prescribing information is generally a piece of paper that includes instructions regarding the proper administration of glycopyrrolate. The prescribing information should be consistent with the methods of treatment described herein.

In particular, the prescribing information can include instructions to a patient or a caregiver of the patient, such as instructions regarding the appropriate age of the patient and the frequency of the administration of the orally disintegrating tablet comprising a therapeutically effective amount of glycopyrrolate. While the patient to be administered the orally disintegrating tablet of the invention can be any suitable age, preferably the patient is older than three years of age (e.g., older than four years of age, older than 5 years of age, older than 6 years of age, older than 7 years of age, older than 8 years of age, older than 9 years of age, older than 10 years of age).

The orally disintegrating tablet can be contained in any suitable container capable of holding and dispensing the orally disintegrating tablet and which will not significantly interact with the orally disintegrating tablet and will further be in physical relation with the prescribing information containing advice regarding the administration of glycopyrrolate. The container may be, for example, a cardboard box or plastic vial. Alternatively, the orally disintegrating tablets may be provided in individual unit dosage forms, such as plastic strips sealed with aluminum foil (e.g., blister package, tear-at-notch, etc.). Such unit dosage forms may optionally be further packaged in a box, vial, or other suitable container along with the prescribing information. When glycopyrrolate is to be administered with a second active agent, the two pharmaceutical dosage forms may be placed in the same or separate containers.

The prescribing information may be associated with the container by any means that maintain a physical proximity of the two. For example, they may both be contained in a packaging material such as a box or plastic shrink wrap. Alternatively, the prescribing information may be bonded to the container such as with suitable glue or other bonding or holding means such that the prescribing information is not obscured.

In another embodiment, the orally disintegrating tablet for oral administration in the methods of the present invention, preferably for buccal delivery systems, comprises an adhesive layer comprising a hydrophilic polymer with one surface adapted to contact a first tissue of the oral cavity and adhere thereto when wet and an opposing surface in contact with and adhering to an adjacent drug/enhancer layer comprising a permeation enhancer and the glycopyrrolate composition. The drug/enhancer layer contacts and is in drug transfer relationship with the buccal mucosa when the adhesive layer contacts and adheres to the first tissue, preferably the gingiva. Typically the hydrophilic polymer comprises compounds selected from the group consisting of hydroxypropyl cellulose, hydroxypropyl methylcellulose, hydroxyethylcellulose, ethylcellulose, carboxymethyl cellulose, dextran, guar-gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and mixtures thereof.

The adhesive layer may additionally contain one or more members including, but not limited to, fillers, tableting excipients, lubricants, flavors, or dyes. The drug/enhancer layer additionally may contain one or members, such as tableting excipients, fillers, flavors, taste-masking agents, dyes, stabilizers, enzyme inhibitors, and lubricants.

The following example further illustrates the invention but, of course, should not be construed as in any way limiting its scope.

### EXAMPLE 1

This example describes the preparation of an orally disintegrating tablet of the invention.

| Component | Amount |
|---|---|
| Glycopyrrolate | 1 mg–10 mg |
| Binder (e.g., gelatin) | 1–20 wt. % |
| Filler (e.g., mannitol) | 1–20 wt. % |
| Sweetener (e.g., aspartame) | 0.001–70 wt. % |
| Flavoring Agent | 0.0001–5 wt. % |
| Preservative (sodium methylhydroxybenzoate and/or | 0.0001–10 wt. % |
| sodium propylhydroxybenzoate) | |
| Purified Water | as necessary* |

*The water is removed during the freeze-drying process

The components of the composition are mixed together as, for example, described herein. The suspension is then poured into blister molds. The suspension is frozen, freeze-dried, and then sealed with a covering sheet adhered to the mold.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An orally disintegrating tablet comprising a therapeutically effective amount of glycopyrrolate.

2. The orally disintegrating tablet of claim 1, wherein the therapeutically effective amount of glycopyrrolate is from about 1 mg to about 10 mg.

3. The orally disintegrating tablet of claim 2, wherein the therapeutically effective amount of glycopyrrolate is from about 1 mg to about 2 mg.

4. The orally disintegrating tablet of claim 1, wherein the therapeutically effective amount of glycopyrrolate is effective for treating sialorrhea.

5. The orally disintegrating tablet of claim 1, wherein said tablet disintegrates in about five minutes or less upon contact with saliva.

6. The orally disintegrating tablet of claim 5, wherein said tablet disintegrates in about three minutes or less upon contact with saliva.

7. The orally disintegrating tablet of claim 6, wherein said tablet disintegrates in about one minute or less upon contact with saliva.

8. A method of treating sialorrhea in a patient, comprising:
identifying a patient demonstrating sialorrhea; and
administering an orally disintegrating tablet comprising a therapeutically effective amount of glycopyrrolate to the patient,
thereby treating sialorrhea in the patient.

9. The method of claim 8, wherein the therapeutically effective amount of glycopyrrolate is from about 1 mg to about 10 mg.

10. The method of claim 9, wherein the therapeutically effective amount of glycopyrrolate is from about 1 mg to about 2 mg.

11. The method of claim 9, wherein the therapeutically effective amount of glycopyrrolate is administered from one to three times daily.

12. The method of claim 8, wherein the patient is older than three years.

13. The method of claim 8, wherein the patient suffers from a neurological dysfunction or disorder.

14. The method of claim 13, wherein the neurological dysfunction or disorder is Parkinson's Disease, stroke, cerebral palsy, amyotrophic lateral sclerosis, or mental retardation.

15. The method of claim 8, wherein the patient suffers from facial paralysis or cancer about the face, neck, or esophagus.

16. A method of treating sialorrhea in a patient, comprising the steps of:
providing to the patient or a caregiver of the patient a kit comprising one or more orally disintegrating tablets comprising a therapeutically effective amount of glycopyrrolate, prescribing information, and a container; and
instructing the patient or the caregiver of the patient regarding administering the orally disintegrating tablet comprising a therapeutically effective amount of glycopyrrolate,
thereby treating sialorrhea in the patient.

17. The method of claim 16, wherein the therapeutically effective amount of glycopyrrolate is about 1 mg to about 10 mg.

18. The method of claim 17, wherein the therapeutically effective amount of glycopyrrolate is about 1 mg to about 2 mg.

19. The method of claim 17, wherein the therapeutically effective amount of glycopyrrolate is administered from one to three times daily.

20. The method of claim 16, wherein the patient is older than three years.

21. The method of claim 16, wherein the patient suffers from a neurological dysfunction or disorder.

22. The method of claim 21, wherein the neurological dysfunction or disorder is Parkinson's Disease, stroke, cerebral palsy, amyotrophic lateral sclerosis, or mental retardation.

23. The method of claim 16, wherein the patient suffers from facial paralysis or cancer about the face, neck, or esophagus.
